(19) United States
(12) Patent Application Publication
(10) Pub. No.: US 2019/0261841 A1
(43) Pub. Date: Aug. 29, 2019

(54) MEDICAL CONTROL APPARATUS, MEDICAL OBSERVATION APPARATUS, AND CONTROL METHOD

(71) Applicant: Sony Olympus Medical Solutions Inc., Tokyo (JP)

(72) Inventor: Shigeru TAMURA, Tokyo (JP)

(73) Assignee: Sony Olympus Medical Solutions Inc., Tokyo (JP)

(21) Appl. No.: 16/258,918

(22) Filed: Jan. 28, 2019

(30) Foreign Application Priority Data
Feb. 28, 2018 (JP) 2018-034550

(51) Int. Cl. A61B 1/05; A61B 34/00; A61B 1/00; A61B 1/005; A61B 90/00

(52) U.S. Cl. CPC A61B 1/05; A61B 34/25; A61B 1/00009; A61B 1/00147; A61B 1/0051; A61B 90/37; A61B 1/00045

(57) ABSTRACT

There is provided a medical control apparatus including: a display control section configured to control a display of a medical captured image captured by an imaging device on the basis of a state related to a movement of the imaging device. In a case in which the state related to the movement of the imaging device is a first state in which the imaging device is immovable, the display control section causes the medical captured image to be displayed on a display screen, and in a case in which the state related to the movement of the imaging device is a second state in which the imaging device is movable, the display control section causes a medical still image to be displayed on the display screen, and changes a display of the display screen according to the movement of the imaging device.

### CROSS REFERENCE TO RELATED APPLICATIONS

This application claims the benefit of Japanese Priority Patent Application JP 2018-034550 filed Feb. 28, 2018, the entire contents of which are incorporated herein by reference.

### BACKGROUND

The present disclosure relates to a medical control apparatus, a medical observation apparatus, and a control method.

Recently, in the medical field, to support microsurgery such as neurosurgical procedures, or to perform an endoscopic surgery, for example, medical observation apparatus capable of enlarged observation of an observation target such as an affected area are used in some cases. Examples of medical observation apparatus include a medical observation apparatus provided with an optical microscope, and a medical observation apparatus provided with an imaging device that functions as an electronic imaging microscope. In the following, the above medical observation apparatus provided with an optical microscope will be designated an “optical medical observation apparatus”. Also, in the following, the above medical observation apparatus provided with an imaging device will be designated an “electronic imaging medical observation apparatus” or simply a “medical observation apparatus” in some cases. Also, in the following, a captured image (a moving image or a still image) in which an observation target is captured by an imaging device provided in a medical observation apparatus is denoted a “medical captured image”.

Among these, technology related to an optical medical observation apparatus that changes an observation image observed in a scope body to 3D/2D in conjunction with the movement/fixation of the scope body is being developed. Examples of the above technology include the technology described in JP 2005-66080A.

### SUMMARY

With an electronic imaging medical observation apparatus, along with the increased image quality of imaging devices, the increased image quality of display apparatus on which captured images are displayed, and the like, the same or higher image quality than an optical medical observation apparatus has come to be obtained. Also, because a user who uses an electronic imaging medical observation apparatus (for example, medical personnel such as a surgeon or a surgeon's assistant; hereinafter simply designated the “user” in some cases) is not required to peer into an eyepiece lens included in an optical microscope like in the case of using an optical medical observation apparatus, it is possible to move the position of the imaging device more freely. For this reason, using an electronic imaging medical observation apparatus has an advantage of enabling more flexible support of surgery by the movement of the position of the imaging device, and in the medical field, utilization of electronic imaging medical observation apparatus is progressing. As above, since the user of an electronic imaging medical observation apparatus is able to move the position of the imaging device freely, the user is able to alter the imaging range by moving the position of the imaging device. At this point, if the case of imaging by an imaging device in a state of a high enlargement ratio is considered, it is anticipated that moving the position of the imaging device will cause an increased amount of shake (what is called image shake) in the medical captured image displayed on the display screen. In the case of an increased amount of shake in the medical captured image in association with the movement of the imaging device, it is difficult for “the user to move the imaging device such that a desired imaging range is captured while also looking at the medical captured image displayed on the display screen”.

Also, since the depth of field is shallow in the state of a high enlargement ratio, for example, even a slight displacement of the imaging device in the dept direction with respect to the surgical site treated as the observation target will cause the medical captured image displayed on the display screen to become a blurry image. Furthermore, in the case in which the medical captured image displayed on the display screen becomes a blurry image, it becomes even more difficult for “the user to move the imaging device such that a desired imaging range is captured while also looking at the medical captured image displayed on the display screen”.

Therefore, in cases in which situations like the above occur in association with the movement of the imaging device, there is a possibility of reduced convenience for the user who uses the medical observation apparatus.

At this point, even if the technology of switching between 3D/2D in conjunction with the movement/fixation of the scope body like the technology described in JP 2005-66080A for example is successfully applied to an electronic imaging medical observation apparatus, situations like the above may still occur. The present disclosure proposes a novel and improved medical control apparatus, medical observation apparatus, and control method capable of achieving an improvement in convenience for the user who uses a medical observation apparatus.

According to an embodiment of the present disclosure, there is provided a medical control apparatus including: a display control section configured to control a display of a medical captured image captured by an imaging device on the basis of a state related to a movement of the imaging device. In a case in which the state related to the movement of the imaging device is a first state in which the imaging device is immovable, the display control section causes the medical captured image to be displayed on a display screen, and in a case in which the state related to the movement of the imaging device is a second state in which the imaging device is movable, the display control section causes a medical still image to be displayed on the display screen, and changes a display of the display screen according to the movement of the imaging device.

In addition, according to an embodiment of the present disclosure, there is provided a medical observation apparatus including: an arm including multiple links joined to each other by one or multiple joint sections; and an imaging device supported by the arm; and a display control section configured to control a display of a medical captured image captured by the imaging device on the basis of a state related to a movement of the imaging device. In a case in which the state related to the movement of the imaging device is a first state in which the imaging device is immovable, the display control section causes the medical captured image to be displayed on a display screen, and in a case in which the state related to the movement of the imaging device is a second state in which the imaging device is movable, the display control section causes a medical still image to be displayed on the display screen, and changes a display of the display screen according to the movement of the imaging device.

In addition, according to an embodiment of the present disclosure, there is provided a control method executed by a medical control apparatus, the control method including: controlling a display of a medical captured image captured by an imaging device on the basis of a state related to a movement of the imaging device. In a case in which the state related to the movement of the imaging device is a first state in which the imaging device is immovable, the controlling causes the medical captured image to be displayed on a display screen, and in a case in which the state related to the movement of the imaging device is a second state in which the imaging device is movable, the controlling causes a medical still image to be displayed on the display screen, and changes a display of the display screen according to the movement of the imaging device.

According to an embodiment of the present disclosure, an improvement in convenience for the user who uses a medical observation apparatus may be achieved. Note that the effects described above are not necessarily limitative. With or in the place of the above effects, there may be achieved any one of the effects described in this specification or other effects that may be grasped from this specification.

### BRIEF DESCRIPTION OF THE DRAWINGS

FIG. 1 is an explanatory diagram illustrating an example of a configuration of a medical observation system according to the present embodiment;

FIG. 2 is an explanatory diagram illustrating an example of a use case in which the medical observation system according to the present embodiment is used;

FIG. 3 is an explanatory diagram for explaining an example of the configuration of an imaging device provided in a medical observation apparatus according to the present embodiment;

FIG. 4 is a function block diagram illustrating one example of a configuration of a medical observation apparatus according to the present embodiment;

FIG. 5 is an explanatory diagram for explaining a first example of processes related to a control method according to the present embodiment;

FIG. 6 is an explanatory diagram for explaining the first example of processes related to a control method according to the present embodiment;

FIG. 7 is an explanatory diagram for explaining a second example of processes related to a control method according to the present embodiment;

FIG. 8 is an explanatory diagram for explaining a third example of processes related to a control method according to the present embodiment; and

FIG. 9 is a flowchart illustrating one example of processes related to the control method according to the present embodiment.

### DETAILED DESCRIPTION OF THE EMBODIMENT(S)

Hereinafter, (a) preferred embodiment(s) of the present disclosure will be described in detail with reference to the appended drawings. In this specification and the appended drawings, structural elements that have substantially the same function and structure are denoted with the same reference numerals, and repeated explanation of these structural elements is omitted.

The description hereinafter will proceed in the following order.

1. Medical observation system according to present embodiment and control method according to present embodiment
[1] Configuration of medical observation system
   [1-1] Display apparatus 200
   [1-2] Medical observation apparatus 100
[2] Control method according to present embodiment
   [2-1] Summary of control method according to present embodiment
   [2-2] Processes related to control method according to present embodiment
   [2-3] Specific example of processes related to control method according to present embodiment
[3] Example of advantageous effects exhibited by use of control method according to present embodiment

2. Program according to present embodiment

### (Medical Observation System According to Present Embodiment and Control Method According to Present Embodiment)

Hereinafter, an example of a medical observation system according to the present embodiment will be described, while a control method according to the present embodiment will also be described.

Hereinafter, the case in which the medical observation apparatus according to the present embodiment executes processes related to the control method according to the present embodiment, that is, the case in which the medical observation apparatus according to the present embodiment functions as a medical control apparatus will be described primarily. Note that in the medical observation system according to the present embodiment, the apparatus that functions as the medical control apparatus is not limited to the medical observation apparatus according to the present embodiment. For example, in the medical observation system according to the present embodiment, the display apparatus described later may also function as the medical control apparatus that executes the processes related to the control method according to the present embodiment. For example, in the medical observation system according to the present embodiment, any apparatus capable of executing the processes related to the control method according to the present embodiment, such as a medical controller, may function as the medical control apparatus.

### [1] Configuration of Medical Observation System

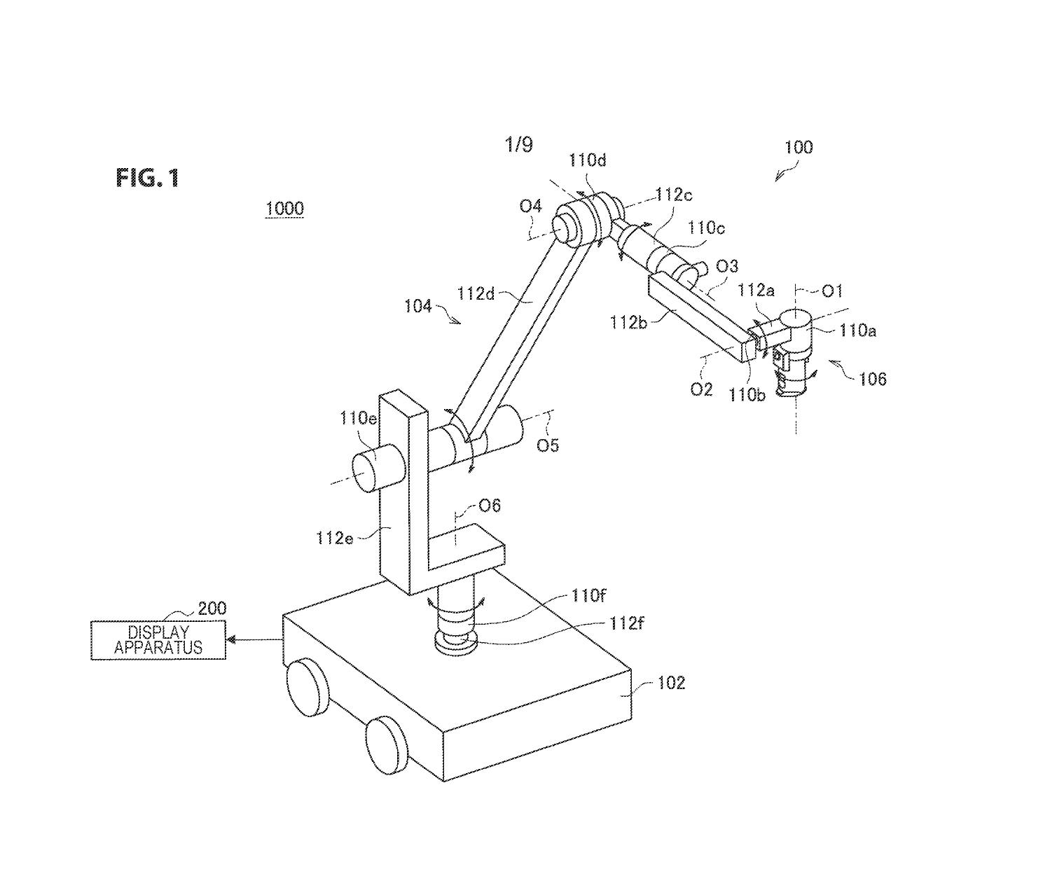

FIG. 1 is an explanatory diagram illustrating an example of the configuration of a medical observation system 1000 according to the present embodiment. The medical observation system 1000 includes a medical observation apparatus 100 and a display apparatus 200, for example.

Note that the medical observation system according to the present embodiment is not limited to the example illustrated in FIG. 1.

For example, the medical observation system according to the present embodiment additionally may include a medical control apparatus (not illustrated) that controls various operations in the medical observation apparatus 100. In the medical observation system 1000 illustrated in FIG. 1, as described later, an example is illustrated in which, by providing the medical observation apparatus 100 with a control section (described later) that performs processes according to the control method according to the present embodiment, the medical observation apparatus 100 includes the functions of the medical control apparatus (not illustrated).

Examples of the medical control apparatus (not illustrated) include arbitrary equipment capable of performing processes according to the control method according to the present embodiment, such as a “medical controller” and a “computer such as a server”. Also, the medical control apparatus (not illustrated) may be, for example, an integrated circuit (IC) that can be embedded in equipment like the above.

Additionally, the medical observation system according to the present embodiment may also be a configuration that includes multiple medical observation apparatuses 100 and display apparatuses 200. In the case of including multiple medical observation apparatuses 100, in each medical observation apparatus 100, processes according to the control method in the medical observation apparatus 100 described later are performed. Also, in the case in which the medical observation system according to the present embodiment is a configuration that includes multiple medical observation apparatuses 100 and display apparatuses 200, the medical observation apparatus 100 and the display apparatus 200 may be associated in a 1-to-1 manner, or multiple medical observation apparatuses 100 may be associated with a single display apparatus 200. In the case in which multiple medical observation apparatuses 100 are associated with a single display apparatus 200, which medical observation apparatus 100 provides a medical captured image to be displayed on a display screen is switched by performing a switching operation or the like in the display apparatus 200, for example.

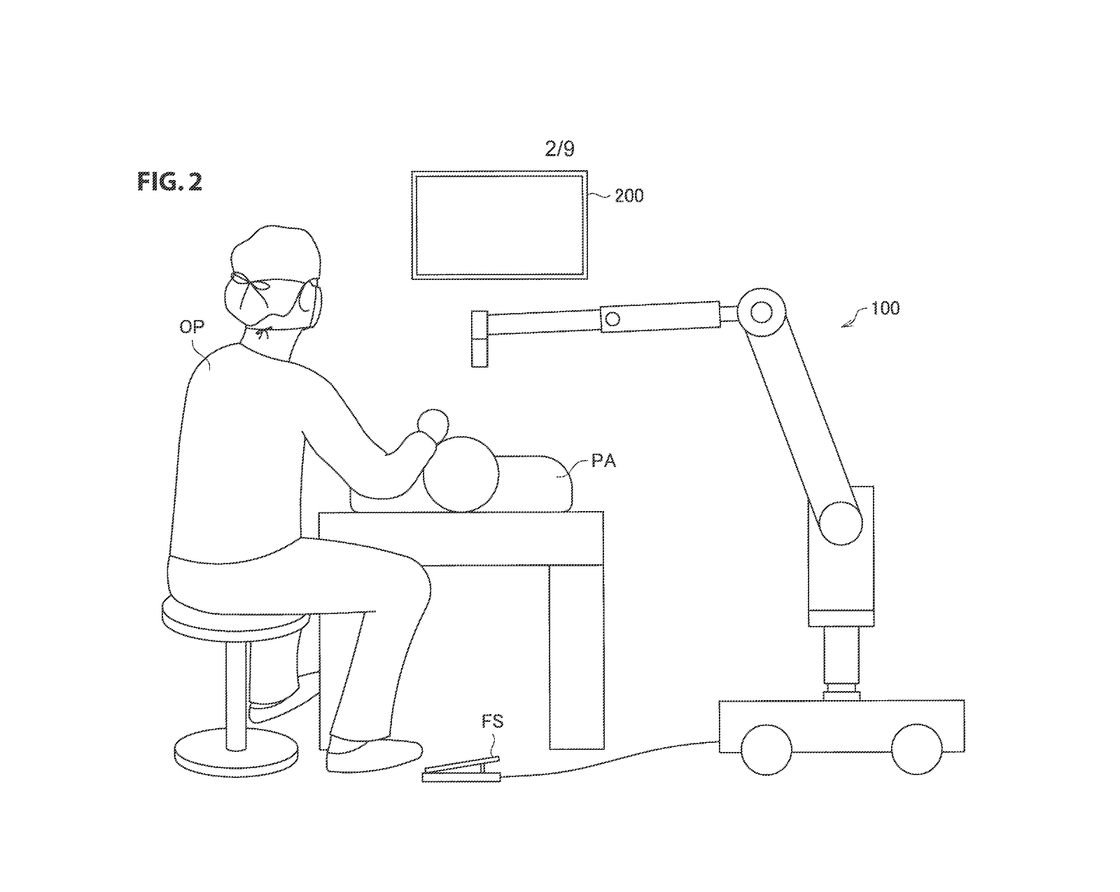

FIG. 2 is an explanatory diagram illustrating an example of a use case in which the medical observation system 1000 according to the present embodiment is used.

By an imaging device (described later) provided in the medical observation apparatus 100, an observation target patient PA (a patient who undergoes a medical procedure) is imaged. A captured image that captures the above patient who undergoes a medical procedure, corresponds to an example of a “medical captured image”.

The medical captured image captured in the medical observation apparatus 100 is displayed on a display screen of a display apparatus 200. Subsequently, a surgeon OP (an example of a user of the medical observation apparatus 100) who performs a medical procedure by using the medical observation apparatus 100 performs the medical procedure on the patient PA while looking at the medical captured image displayed on the display screen of the display apparatus 200.

Also, the surgeon OP operates an operating device external to the medical observation apparatus 100, such as a footswitch FS, or an operating device (described later) provided in the medical observation apparatus 100, thereby causing an arm (described later) and the imaging device (described later) provided in the medical observation apparatus 100 to operate, and putting the medical observation apparatus 100 into a desired state.

Hereinafter, each apparatus included in the medical observation system 1000 will be described.

### [1-1] Display Apparatus 200

The display apparatus 200 is a display device in the medical observation system 1000, and corresponds to an external display device from the perspective of the medical observation apparatus 100. The display apparatus 200 displays various images on a display screen, such as a medical captured image (a moving image or still images; the same applies hereinafter) taken in the medical observation apparatus 100, or an image related to a user interface (UI), for example. Hereinafter, a medical captured image that is a still image will be designated a “medical still image” in some cases. Also, the display apparatus 200 may include a configuration capable of 3D display according to any method. The display on the display apparatus 200 is controlled by, for example, the medical observation apparatus 100 or the medical control apparatus (not illustrated).

In the medical observation system 1000, the display apparatus 200 is installed in an arbitrary location visible to a person involved in a surgery inside an operating room, such as on a wall, the ceiling, or the floor of the operating room. Examples of the display apparatus 200 include a liquid crystal display, an organic electro-luminescence (EL) display, a cathode ray tube (CRT) display, and the like.

Note that the display apparatus 200 is not limited to the example illustrated above.

For example, the display apparatus 200 may also be an arbitrary wearable apparatus that is used by being worn on the body of the surgeon or the like, such as a head-mounted display, an eyewear-type apparatus, or the like.

The display apparatus 200 runs on electric power supplied from an internal power source such as a battery provided in the display apparatus 200, on electric power supplied from a connected external power source, or the like, for example.

### [1-2] Medical Observation Apparatus 100

The medical observation apparatus 100 is an electronic imaging medical observation apparatus. For example, in the case in which the medical observation apparatus 100 is used during surgery, the surgeon (one example of the user of the medical observation apparatus 100) observes an operating site (an affected area) while referring to a medical captured image which has been taken by the medical observation apparatus 100 and displayed on the display screen of the display apparatus 200, and performs various treatments, such as techniques depending on the surgical procedure, on the operating site.

As illustrated in FIG. 1, the medical observation apparatus 100 is provided with a base 102, an arm 104, and an imaging device 106, for example.

Additionally, although not illustrated in FIG. 1, the medical observation apparatus 100 may also be provided with, for example, one or multiple processors (not illustrated) including a computational circuit such as a microprocessing unit (MPU), read-only memory (ROM; not illustrated), random access memory (RAM; not illustrated), a recording medium (not illustrated), and a communication device (not illustrated). The medical observation apparatus 100 runs on electric power supplied from an internal power source such as a battery provided in the medical observation apparatus 100, on electric power supplied from a connected external power source, or the like, for example.

The one or multiple processors (not illustrated) function as the control section in the medical observation apparatus 100 (described later). The ROM (not illustrated) stores programs and control data such as computational parameters used by the one or multiple processors (not illustrated). The RAM (not illustrated) temporarily stores programs executed by the one or multiple processors (not illustrated), or the like.

The recording medium (not illustrated) functions as a storage section (not illustrated) in the medical observation apparatus 100. A variety of data is stored on the recording medium (not illustrated), including data related to the control method according to the present embodiment, and various applications, for example. Herein, examples of the recording medium (not illustrated) include a magnetic recording medium such as a hard disk, non-volatile memory such as flash memory, and the like. Additionally, the recording medium (not illustrated) may also be removable from the medical observation apparatus 100.

The communication device (not illustrated) is a communication device provided in the medical observation apparatus 100, and fulfills a role of communicating in a wireless or wired manner with an external apparatus such as the display apparatus 200. Herein, examples of the communication device (not illustrated) include an IEEE 802.15.1 port and transmitting-receiving circuit (wireless communication), an IEEE 802.11 port and transmitting-receiving circuit (wireless communication), a communication antenna and a RF circuit (wireless communication), a LAN terminal and a transmitting-receiving circuit (wired communication), and the like.

### [1-2-1] Base 102

The base 102 is the base of the medical observation apparatus 100. One end of the arm 104 is connected to the base 102, and the base 102 supports the arm 104 and the imaging device 106.

Also, casters are provided on the base 102, for example, and the medical observation apparatus 100 contacts the floor through the casters. By providing the casters, the medical observation apparatus 100 is able to move easily over the floor by the casters.

### [1-2-2] Arm 104

The arm 104 includes multiple links joined to each other by joint sections. In addition, the arm 104 supports the imaging device 106. The imaging device 106 supported by the arm 104 is movable three-dimensionally, and after moving, the position and the attitude of the imaging device 106 are maintained by the arm 104.

More specifically, the arm 104 includes, for example, multiple joint sections 110a, 110b, 110c, 110d, 110e, and 110f, and multiple links 112a, 112b, 112c, 112d, 112e, and 112f rotatably joined to each other by the joint sections 110a, 110b, 110c, 110d, 110e, and 110f. The rotatable range of each of the joint sections 110a, 110b, 110c, 110d, 110e, and 110f is set arbitrarily during the design stage, the manufacturing stage, or the like so that the desired motion of the arm 104 is realized.

In other words, in the medical observation apparatus 100 illustrated in FIG. 1, six degrees of freedom are realized in relation to the movement of the imaging device 106 by six rotation axes (first axis O1, second axis O2, third axis O3, fourth axis O4, fifth axis O5, and sixth axis O6: an example of a prescribed rotation axis in the medical observation apparatus 100) corresponding to the six joint sections 110a, 110b, 110c, 110d, 110e, and 110f included in the arm 104. More specifically, in the medical observation apparatus 100 illustrated in FIG. 1, motion with six degrees of freedom, including three degrees of translational freedom and three degrees of rotational freedom, is realized.

Actuators (not illustrated) are provided in each of the joint sections 110a, 110b, 110c, 110d, 110e, and 110f. Each of the joint sections 110a, 110b, 110c, 110d, 110e, and 110f rotates about the corresponding rotation axis by the driving of the actuators (not illustrated). The driving of the actuators (not illustrated) is controlled by, for example, a processor that functions as the control section described later, or an external medical control apparatus (not illustrated).

Each of the joint sections 110a, 110b, 110c, 110d, 110e, and 110f may be provided with sensors capable of detecting a rotational angle for each of six rotation axes (one example of predetermined rotation axes; the same applies hereinafter). The sensors according to the present embodiment may be, for example, angle sensors such as rotary encoders, or any sensors capable of obtaining a rotational angle for each of six rotation axes, such as angular velocity sensors.

By having each of the joint sections 110a, 110b, 110c, 110d, 110e, 110f rotate about the corresponding rotation axis by the driving of the actuators (not illustrated), various operations of the arm 104, such as extending and contracting (folding up) the arm 104, for example, are realized.

The joint section 110a has an approximately cylindrical shape, and supports the imaging device 106 (the top end of the imaging device 106 in FIG. 1) on the front end portion of the joint section 110a (the bottom end portion in FIG. 1), so as to allow revolution about a rotation axis (first axis O1) parallel to the central axis of the imaging device 106. Herein, the medical observation apparatus 100 is configured so that the first axis O1 is aligned with the optical axis in the imaging device 106. In other words, by causing the imaging device 106 to revolve about the first axis O1 illustrated in FIG. 1, the medical captured image captured by the imaging device 106 becomes an image which has changed so that the field of view rotates.

The link 112a is an approximately rod-shaped member, and securely supports the joint section 110a. The link 112a extends in a direction orthogonal to the first axis O1, for example, and is connected to the joint section 110b.

The joint section 110b has an approximately cylindrical shape, and supports the link 112a so as to allow revolution about a rotation axis (second axis O2) orthogonal to the first axis O1. Also, the link 112b is securely connected to the joint section 110b.

The link 112b is an approximately rod-shaped member, and extends in a direction orthogonal to the second axis O2. Also, each of the joint section 110b and the joint section 110c is connected to the link 112b.

The joint section 110c has an approximately cylindrical shape, and supports the link 112b so as to allow revolution about a rotation axis (third axis O3) mutually orthogonal to each of the first axis O1 and the second axis O2. Also, one end of the link 112c is securely connected to the joint section 110c.

Herein, by having the front end side (the side on which the imaging device 106 is provided) of the arm 104 revolve about the second axis O2 and the third axis O3, the imaging device 106 can be made to move so that the position of the imaging device 106 in the horizontal plane is changed. In other words, in the medical observation apparatus 100, controlling the rotation about the second axis O2 and the third axis O3 makes it possible to move the field of view of the medical captured image in a flat plane.

The link 112c is a member in which one end has an approximately cylindrical shape, and the other end has an approximately rod-like shape. On the side of the one end of the link 112c, the joint section 110c is securely connected so that the central axis of the joint section 110c and the central axis of the approximately cylindrical shape are the same. Also, on the side of the other end of the link 112c, the joint section 110d is connected.

The joint section 110d has an approximately cylindrical shape, and supports the link 112c so as to allow revolution about a rotation axis (fourth axis O4) orthogonal to the third axis O3. The link 112d is securely connected to the joint section 110d.

The link 112d is an approximately rod-shaped member, and extends orthogonally to the fourth axis O4. One end of the link 112d is securely connected to the joint section 110d so as to abut the approximately cylindrical side face of the joint section 110d. Also, the joint section 110e is connected to the other end of the link 112d (the end on the opposite side of the side where the joint section 110d is connected).

The joint section 110e has an approximately cylindrical shape, and supports one end of the link 112d so as to allow revolution about a rotation axis (fifth axis O5) parallel to the fourth axis O4. Also, one end of the link 112e is securely connected to the joint section 110e.

Herein, the fourth axis O4 and the fifth axis O5 are rotation axis about which the imaging device 106 may be moved in the vertical direction. By having the front end side (the side on which the imaging device 106 is provided) of the arm 104 revolve about the fourth axis O4 and the fifth axis O5, the position of the imaging device 106 in the vertical direction changes. Thus, by having the front end side (the side on which the imaging device 106 is provided) of the arm 104 revolve about the fourth axis O4 and the fifth axis O5, changing the distance between the imaging device 106 and an observation target, such as an operating site of a patient, becomes possible.

The link 112e is a member that includes a combination of a first member having an approximate L-shape with one edge extending in the vertical direction while the other edge extends in the horizontal direction, and a rod-like second member that extends vertically downward from the part of the first member that extends in the horizontal direction. The joint section 110e is securely connected to the part of the first member of the link 112e that extends in the vertical direction. Also, the joint section 110f is connected to the second member of the link 112e. The joint section 110f has an approximately cylindrical shape, and supports the link 112e so as to allow revolution about a rotation axis (sixth axis O6) parallel to the vertical direction. Also, the link 112f is securely connected to the joint section 110f.

The link 112f is an approximately rod-shaped member, and extends in the vertical direction. The joint section 110f is connected to one end of the link 112f. Also, the other end of the link 112f (the end on the opposite side of the side where the joint section 110f is connected) is securely connected to the base 102.

By having the arm 104 include the configuration indicated above, in the medical observation apparatus 100, six degrees of freedom are realized with respect to the movement of the imaging device 106.

Note that the configuration of the arm 104 is not limited to the example indicated above.

For example, each of the joint sections 110a, 110b, 110c, 110d, 110e, and 110f of the arm 104 may be provided with a brake that restrains rotation in each of the joint sections 110a, 110b, 110c, 110d, 110e, and 110f. The brake according to the present embodiment may be a brake of an arbitrary method, such as a mechanically driven brake or an electrically driven electromagnetic brake, for example.

The driving of the above brakes is controlled by, for example, a processor that functions as the control section described later, or an external medical control apparatus (not illustrated). By controlling the driving of the above brakes, in the medical observation apparatus 100, the operating mode of the arm 104 is set. Examples of operating modes of the arm 104 include a locked mode and a free mode.

Herein, the locked mode according to the present embodiment is, for example, an operating mode in which the position and the attitude of the imaging device 106 are locked by using brakes to restrain rotation about each rotation axis provided in the arm 104. By having the arm 104 enter the locked mode, the operating state of the medical observation apparatus 100 becomes a locked state in which the position and the attitude of the imaging device 106 are locked.

Also, the free mode according to the present embodiment is an operating mode in which the above brakes are released, thereby allowing each rotation axis provided in the arm 104 to rotate freely. For example, in the free mode, the position and the attitude of the imaging device 106 are adjustable by direct operations performed by the surgeon. Herein, a direct operation according to the present embodiment means, for example, an operation in which the surgeon grips the imaging device 106 with his or her hand, and directly moves the imaging device 106.

For example, by switching the operating mode of the arm 104 as above, the imaging device 106 supported by the arm 104 is switched between a state in which the imaging device 106 is immovable (hereinafter designated the “first state”) and a state in which the imaging device 106 is movable (hereinafter designated the “second state”). To give a specific example, in the case in which the operating mode of the arm 104 is the locked mode, the imaging device 106 enters the first state, and in the case in which the operating mode of the arm 104 is the free mode, the imaging device 106 enters the second state.

As above, each of the first state and the second state is a state indicating whether or not the imaging device 106 is movable. Hereinafter, the first state and the second state will be collectively designated the “state related to the movement of the imaging device 106” in some cases. Also, hereinafter, a case in which the state related to the movement of the imaging device 106 changes by switching the operating mode of the arm 104 will be given as an example.

### [1-2-3] Imaging Device 106

The imaging device 106 is supported by the arm 104, and images an observation target such as an operating site of a patient, for example. Imaging in the imaging device 106 is controlled by, for example, a processor that functions as the control section described later, or an external medical control apparatus (not illustrated).

The imaging device 106 has a configuration corresponding to an electronic imaging microscope, for example.

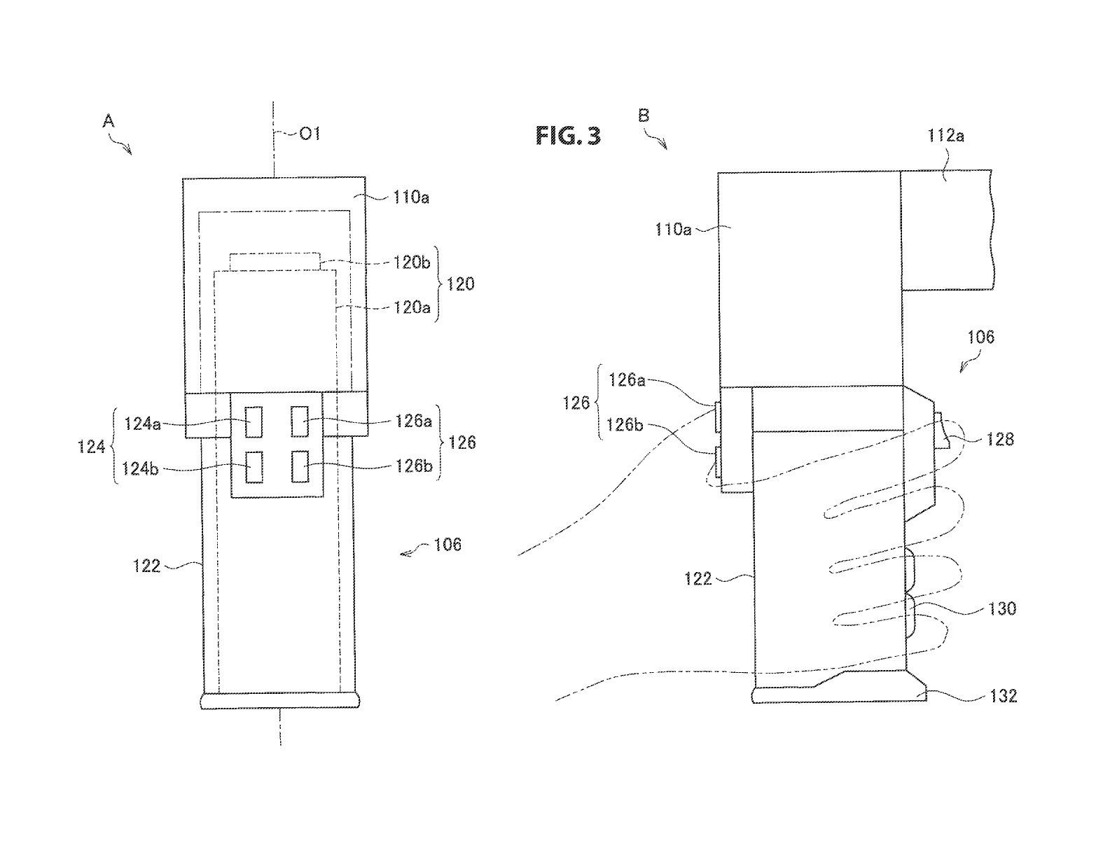

FIG. 3 is an explanatory diagram for explaining an example of the configuration of the imaging device 106 provided in the medical observation apparatus 100 according to the present embodiment.

For example, the imaging device 106 includes an imaging member 120 and a barrel member 122 having an approximately cylindrical shape, with the imaging member 120 being provided inside the barrel member 122.

On an aperture on the bottom end of the barrel member 122 (the lower end in FIG. 3), for example, a cover glass (not illustrated) for protecting the imaging member 120 is provided.

Additionally, for example, a light source (not illustrated) is provided inside the barrel member 122, and during imaging, the subject is irradiated with illuminating light radiating from the light source through the cover glass. Reflected light (observation light) from the subject irradiated with illuminating light enters the imaging member 120 through the cover glass (not illustrated), whereby an image signal indicating the subject (an image signal indicating a medical captured image) is obtained by the imaging member 120.

As the imaging member 120, any of various known types of configurations used in an electronic imaging microscope section can be applied.

To give one example, the imaging member 120 includes an optical system 120a and an image sensor 120b including an imaging element that takes an image of an observation target with light transmitted through the optical system 120a, for example. The optical system 120a includes optical elements such as a mirror and one or multiple lenses, such as an objective lens, a zoom lens, and a focus lens, for example. Examples of the image sensor 120b include an image sensor using multiple imaging elements, such as a complementary metal-oxide semiconductor (CMOS) or a charge-coupled device (CCD).

The imaging member 120, by including two or more imaging devices provided with an optical system 120a and an image sensor 120b, functions as what is called a stereo camera.

Also, in the case in which the imaging member 120 has a configuration including two or more imaging devices, a subset of the imaging devices may also be an imaging device that executes imaging at actual size (or an imaging device that executes imaging at a lower enlargement ratio than the enlargement ratio for normal enlarged observation). The above subset of the imaging devices functions as a sub-camera in the imaging device 106.

Each imaging device included in the imaging member 120 is equipped with one or multiple functions typically provided in an electronic imaging microscope section, such as a zoom function (one or both of an optical zoom function and an electronic zoom function) and an autofocus (AF) function.

In addition, the imaging member 120 may also be configured to be capable of imaging at what are called high resolutions, such as 4K and 8K, for example. By configuring the imaging member 120 to be capable of imaging at high resolutions, it becomes possible to ensure a predetermined resolution (such as full HD image quality, for example), while also displaying an image on the display apparatus 200 having a large display screen, such as 50 inches or more, for example. For this reason, visibility is improved for the surgeon watching the display screen. Also, by configuring the imaging member 120 to be capable of imaging at high resolutions, even if the captured image is enlarged by the electronic zoom function and displayed on the display screen of the display apparatus 200, it is still possible to ensure a predetermined resolution. Furthermore, in the case of using the electronic zoom function to ensure a predetermined resolution, since it is possible to reduce the performance of the optical zoom function in the imaging device 106, the optical system of the imaging device 106 can be simplified, and the imaging device 106 can be configured more compactly.

In the imaging device 106, for example, various operating devices for controlling the operation of the imaging device 106 are provided. For example, in FIG. 3, a zoom switch 124, a focus switch 126, and an operating mode change switch 128 are provided on the imaging device 106. Note that the positions and shapes in which to provide the zoom switch 124, the focus switch 126, and the operating mode change switch 128 obviously are not limited to the example illustrated in FIG. 3. The zoom switch 124 and the focus switch 126 are an example of an operating device for adjusting the imaging parameters in the imaging device 106.

The zoom switch 124 includes, for example, a zoom-in switch 124a that increases the zoom magnification (enlargement ratio), and a zoom-out switch 124b that decreases the zoom magnification. By performing an operation on the zoom switch 124, the zoom magnification is adjusted, and the zoom is adjusted.

The focus switch 126 includes, for example, a long-range focus switch 126a that increases the focal length to the observation target (subject), and a close-range focus switch 126b that decreases the focal length to the observation target. By performing an operation on the focus switch 126, the focal length is adjusted, and the focus is adjusted.

The operating mode change switch 128 is an example of an operating device for changing the operating mode of the arm 104 in the imaging device 106. By performing an operation on the operating mode change switch 128, the operating mode of the arm 104 is changed. Examples of operating modes of the arm 104 include a locked mode and a free mode, as described above.

One example of an operation with respect to the operating mode change switch 128 is an operation of pressing the operating mode change switch 128. For example, the operating mode of the arm 104 becomes the free mode while the surgeon is pressing the operating mode change switch 128, and the operating mode of the arm 104 becomes the locked mode when the surgeon is not pressing the operating mode change switch 128.

By performing an operation on the operating mode change switch 128 and changing the operating mode of the arm 104, the state related to the movement of the imaging device 106 is switched between the first state and the second state.

In addition, the imaging device 106 is provided with, for example, an anti-slip member 130 and a projecting member 132 in order to further raise operability, convenience, and the like when an operator who performs operations on various operation devices performs an operation.

The anti-slip member 130 is a member provided to prevent slipping of an operating body such as a hand when, for example, the operator performs an operation on the barrel member 122 with the operating body. The anti-slip member 130 is formed with a material having a large coefficient of friction, for example, and has a slip-resistant structure due to unevenness or the like.

The projecting member 132 is member provided to prevent an operating body such as a hand blocking the field of view of the optical system 120a when the operator performs an operation on the barrel member 122 with the operating body, or to prevent a cover glass (not illustrated) from becoming dirty due to the cover glass being contacted by the operating body when an operation is performed with the operating body.

Note that the position and shape in which each of the anti-slip member 130 and the projecting member 132 is provided obviously are not limited to the example illustrated in FIG. 3. In addition, the imaging device 106 does not have to be provided with one or both of the anti-slip member 130 and the projecting member 132.

The image signal (image data) generated by imaging in the imaging device 106 is subjected to image processing in a processor that functions as the control section described later, for example. Examples of image processing according to the present embodiment include one or multiple processes from among various processes such as gamma correction, white balance adjustment, image enlargement or reduction related to the electronic zoom function, and pixel interpolation, for example. Note that in the case in which the medical observation system according to the present embodiment includes a medical control apparatus (not illustrated) that controls various operations in the medical observation apparatus 100, the image processing according to the present embodiment may also be performed in the medical control apparatus (not illustrated).

For example, the medical observation apparatus 100 transmits a display control signal and the image signal subjected to imaging processing as above to the display apparatus 200.

By transmitting the display control signal and the image signal to the display apparatus 200, on the display screen of the display apparatus 200, a medical captured image in which the observation target is imaged (for example, a captured image in which the operating site is imaged) is displayed enlarged or reduced at a desired magnification by one or both of the optical zoom function and the electronic zoom function.

The medical observation apparatus 100 includes the hardware configuration illustrated with reference to FIGS. 1 and 3, for example.

Note that the hardware configuration of the medical observation apparatus according to the present embodiment is not limited to the configuration illustrated with reference to FIGS. 1 and 3.

For example, the medical observation apparatus according to the present embodiment may also be a configuration not provided with the base 102, in which the arm 104 is directly attached to the ceiling, a wall, or the like of the operating room or the like. For example, in the case in which the arm 104 is attached to the ceiling, the medical observation apparatus according to the present embodiment becomes a configuration in which the arm 104 hangs down from the ceiling.

Also, although FIG. 1 illustrates an example in which the arm 104 is configured so that six degrees of freedom are realized with respect to the driving of the imaging device 106, the configuration of the arm 104 is not limited to a configuration whereby the degrees of freedom with respect to the driving of the imaging device 106 become six degrees of freedom. For example, it is sufficient to configure the arm 104 so that the imaging device 106 can move appropriately in accordance with the application, and factors such as the number and arrangement of joint sections and links, and the directions of the drive shafts of the joint sections can be set appropriately so that the arm 104 has the desired degrees of freedom.

Also, although FIGS. 1 and 3 illustrate an example in which various types of operating devices for controlling the operation of the imaging device 106 are provided on the imaging device 106, some or all of the operating devices illustrated in FIGS. 1 and 3 may also not be provided on the imaging device 106. To give one example, the various types of operating devices for controlling the operation of the imaging device 106 may also be provided in another part other than the imaging device 106 included in the medical observation apparatus according to the present embodiment. Also, to give another example, the various types of operating devices for controlling the operation of the imaging device 106 may also be external operating devices, such as a footswitch or a remote controller.

Additionally, the imaging device 106 may also have a configuration enabling switching among multiple observation modes. Observation modes according to the present embodiment may include, for example, an observation mode that executes imaging with natural light, an observation mode that executes imaging with special light, an observation mode that executes imaging by utilizing an image-enhancing observation technology such as narrow-band imaging (NBI), and the like. Special light according to the present embodiment refers to light in a specific wavelength band, such as light in the fluorescent wavelength band of fluorescent observation using 5-Aminolevulinic acid (5-ALA).

One example of the configuration of the imaging device 106 enabling switching among multiple observation modes is a “configuration provided with a filter that allows light of a specific wavelength band to pass through while not allowing light of other wavelength bands to pass through, and a movement mechanism that selectively disposes the filter on the optical path”, for example. The specific wavelength band that the filter according to the present embodiment allows to pass through may be, for example, the wavelength band of near-infrared rays (for example, the wavelength band from approximately 0.7 [micrometers] to 2.5 [micrometers]), the fluorescent wavelength band for fluorescent observation using 5-ALA (for example, the wavelength band from approximately 0.6 [micrometers] to 0.65 [micrometers]), the fluorescent wavelength band of indocyanine green (ICG) (for example, the wavelength band from approximately 0.82 [micrometers] to 0.85 [micrometers]), or the like.

Note that the imaging device 106 may also be provided with multiple filters that allow different wavelength bands to pass through. Also, although the above illustrates an example in which imaging is executed with the light of a specific wavelength band by disposing a filter on the optical path, the configuration of the imaging device 106 for executing imaging with the light of a specific wavelength band obviously is not limited to the example illustrated above.

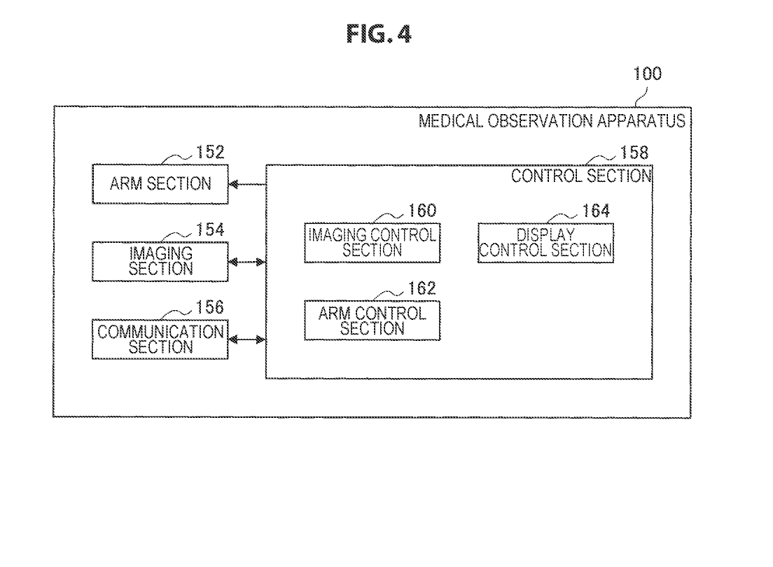

Next, the medical observation apparatus 100 illustrated in FIG. 1 will be described using function blocks. FIG. 4 is a function block diagram illustrating one example of the configuration of the medical observation apparatus 100 according to the present embodiment.

For example, the medical observation apparatus 100 is provided with an arm section 152, an imaging section 154, a communication section 156, and a control section 158.

The arm section 152 includes the arm 104, and supports the imaging device 106 included in the imaging section 154.

The imaging section 154 includes the imaging device 106, and images an observation target. Imaging in the imaging section 154 is controlled by the control section 158, for example.

The communication section 156 is a communication device provided in the medical observation apparatus 100, and fulfills a role of communicating in a wireless or wired manner with an external apparatus such as the display apparatus 200. The communication section 156 includes the communication device (not illustrated) described above, for example. Communication in the communication section 156 is controlled by the control section 158, for example.

The control section 158 includes the processor (not illustrated) described above, for example, and fulfills a role of controlling the medical observation apparatus 100 overall. In addition, the control section 158 fulfills a role of leading the execution of the processes related to the control method described later. Note that the processes related to the control method in the control section 158 may also be executed in a distributed manner by multiple processing circuits (such as multiple processors, for example).

More specifically, the control section 158 includes an imaging control section 160, an arm control section 162, and a display control section 164, for example.

The imaging control section 160 controls the imaging device 106 included in the imaging section 154. Examples of the control of the imaging device 106 include control of one or multiple functions typically provided in an electronic imaging microscope section, such as control of a AF function, including at least a zoom function (one or both of an optical zoom function and an electronic zoom function).

The arm control section 162 controls the driving of the arm 104 included in the arm section 152. One example of control of the driving of the arm 104 includes, for example, “applying a control signal that controls driving to the actuators (not illustrated) corresponding to each of the joint sections 110a, 110b, 110c, Hod, 110e, and 110f”, and the like.

For example, the display control section 164 controls the display on the display apparatus 200 by conveying the display control signal and the image signal to the communication device (not illustrated) included in the communication section 156, and causing the display control signal and the image signal to be transmitted to the display apparatus 200. Note that the control of communication in the communication section 156 may also be performed by a communication control section (not illustrated) included in the control section 158.

Also, the display control section 164 executes the processes related to the control method described later, and controls the display of the medical captured image on the basis of the state related to the movement of the imaging device 106. One example of the processes related to the control method according to the present embodiment and one example of the display of a medical captured image realized by the processes related to the control method according to the present embodiment will be described later.

For example, by including the display control section 164, the control section 158 fulfills a role of leading the execution of the processes related to the control method according to the present embodiment. Also, for example, by including the imaging control section 160, the arm control section 162, and the display control section 164, the control section 158 fulfills a role of controlling the medical observation apparatus 100 overall.

Note that the functional configuration of the control section 158 is not limited to the example illustrated in FIG. 4.

For example, it is possible for the control section 158 to have any configuration corresponding to how the functions included in the medical observation apparatus 100 are divided up, such as a configuration corresponding to how the processes related to the control method according to the present embodiment are divided up.

The medical observation apparatus 100 executes the processes related to the control method according to the present embodiment described later with the configuration illustrated in FIG. 4, for example.

Note that the functional configuration of the medical observation apparatus according to the present embodiment is not limited to the configuration illustrated in FIG. 4.

For example, in the medical observation apparatus according to the present embodiment, some or all of the imaging control section 160, the arm control section 162, and the display control section 164 illustrated in FIG. 4 can be provided separately from the control section 158 (for example, realized by a different processing circuit).

Additionally, in the medical observation apparatus according to the present embodiment, the functional configuration for realizing the processes related to the control method according to the present embodiment is not limited to the configuration illustrated in FIG. 4, and it is possible for the medical observation apparatus according to the present embodiment to take a functional configuration corresponding to how the processes related to the control method according to the present embodiment are divided up.

Also, for example, in the case of communicating with an external apparatus via an external communication device having a function and configuration similar to the communication section 156, the medical observation apparatus according to the present embodiment may also not be provided with the communication section 156.

Also, in the case in which the medical observation system according to the present embodiment includes a medical control apparatus (not illustrated), and the medical observation apparatus according to the present embodiment is controlled by the medical control apparatus (not illustrated), the medical observation apparatus according to the present embodiment may also not be provided with the control section 158.

Herein, the medical control apparatus (not illustrated) is, for example, provided with a control section having a function and configuration similar to the control section 158, and thereby executes processes related to the control method according to the present embodiment described later, and in addition, controls the operation in each structural element such as the arm section 152 and the imaging section 154 provided in the medical observation apparatus according to the present embodiment. The medical control apparatus (not illustrated) communicates with the medical observation apparatus according to the present embodiment via a provided communication device or a connected external communication device, and thereby controls the operation in each structural element provided in the medical observation apparatus according to the present embodiment.

Furthermore, in the case in which the medical observation system according to the present embodiment includes the medical control apparatus (not illustrated), and the medical observation apparatus according to the present embodiment is controlled by the medical control apparatus (not illustrated), it is also possible for the medical observation apparatus according to the present embodiment to take a configuration that does not include some of the functions of the control section 158.

### [2] Control Method According to Present Embodiment

Next, the control method according to the present embodiment will be described. The following gives an example of a case in which the processes related to the control method according to the present embodiment are executed by the medical observation apparatus 100 (more specifically, the control section 158 included in the medical observation apparatus 100, for example). Note that, as described above, in the medical observation system according to the present embodiment, the processes related to the control method according to the present embodiment may also be executed by a medical control apparatus (not illustrated).

### [2-1] Summary of Control Method According to Present Embodiment

In the case in which the operating mode of the arm 104 is the free mode (in the case in which the imaging device 106 is in the second state), the user of the medical observation apparatus 100 is able to move the position of the imaging device 106 freely. As described above, the imaging device 106 is able to change the enlargement ratio (zoom magnification). For this reason, when considering the case in which imaging is being executed by the imaging device 106 in a state of a high enlargement ratio, situations like the following, for example, may occur in association with the movement of the position of the imaging device 106.
Shake (image shake) in the medical captured image displayed on the display screen of the display apparatus 200 becomes larger
Even a slight displacement of the position of the imaging device 106 in the depth direction with respect to the observation target such as a surgical site causes the medical captured image displayed on the display screen of the display apparatus 200 to become a blurry image

In the case of an increased amount of shake in the medical captured image in association with the movement of the imaging device 106, it is difficult for “the user to move the imaging device 106 such that a desired imaging range is captured while also looking at the medical captured image displayed on the display screen”. Also, in the case of increased shake in the medical captured image in association with the movement of the imaging device 106, there is a possibility that the user will feel dizzy and disoriented. Furthermore, in the case in which the medical captured image displayed on the display screen becomes a blurry image, it becomes even more difficult for “the user to move the imaging device 106 such that a desired imaging range is captured while also looking at the medical captured image displayed on the display screen”.

Therefore, in cases in which situations like the above occur in association with the movement of the imaging device 106, there is a possibility of reduced convenience for the user who uses the medical observation apparatus 100.

Accordingly, the medical observation apparatus 100 controls the display of the medical captured image on the basis of the state related to the movement of the imaging device 106, and thereby avoids situations like the above that may occur in association with the movement of the imaging device 106. Also, by avoiding situations like the above that may occur in association with the movement of the imaging device 106, convenience potentially may be improved for the user who uses the medical observation apparatus 100.

### [2-2] Processes Related to Control Method According to Present Embodiment

Next, the processes related to the control method according to the present embodiment will be described more specifically.

The medical observation apparatus 100 controls the display of the medical captured image on the basis of the state related to the movement of the imaging device 106. More specifically, the medical observation apparatus 100 executes any from the processes related to a first example illustrated in (1) to the processes related to a third example illustrated in (3) below as the processes related to the control method according to the present embodiment.

### (1) First Example of Processes Related to Control Method

The medical observation apparatus 100 causes the medical captured image to be displayed on the display screen in the case in which the state related to the movement of the imaging device 106 is the first state in which the imaging device 106 is immovable.

Also, in the case in which the medical observation apparatus 100 is in the state in which the imaging device 106 is movable, the medical observation apparatus 100 causes a medical still image (one example of a medical captured image) to be displayed on the display screen.

For example, in the case in which the state related to the movement of the imaging device 106 changes from the first state to the second state, the medical observation apparatus 100 causes the medical still image to be displayed on the display screen.

The medical observation apparatus 100 specifies whether the state related to movement is the first state or the state related to movement is the second state on the basis of a result of detecting a predetermined switching operation that switches between the first state and the second state. The predetermined switching operation may be, for example, an operation on the operating mode change switch 128, or an operation on an external operating device such as a remote controller.

The medical still image according to the first example of the processes related to the control method is a still image of the medical captured image captured when changing from the first state to the second state. Examples of the still image of the medical captured image captured when changing from the first state to the second state include the examples illustrated below.
A still image captured by the imaging device 106 at the point in time when the predetermined switching operation is detected
A frame image included in a moving image captured by the imaging device 106 at the point in time when the predetermined switching operation is detected
A still image captured by the imaging device 106 after the predetermined switching operation is detected but before movement of the imaging device 106 is detected
A frame image included in a moving image captured by the imaging device 106 after the predetermined switching operation is detected but before movement of the imaging device 106 is detected

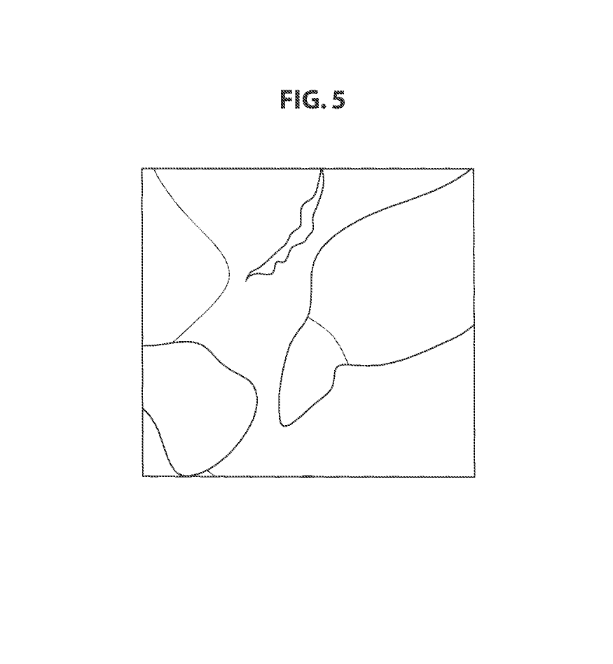

FIG. 5 is an explanatory diagram for explaining the first example of the processes related to the control method according to the present embodiment, and illustrates one example of a medical still image according to the first example of the processes related to the control method.

When the medical still image is displayed on the display screen, the medical observation apparatus 100 changes the display of the display screen according to the movement of the imaging device 106. For example, the medical observation apparatus 100 changes the display of the display screen according to the movement of the imaging device 106 by altering the display of the medical still image displayed on the display screen to correspond with the movement of the imaging device 106.

Altering the display of the medical still image to correspond with the movement of the imaging device 106 refers to, for example, “altering the display of the medical still image such that the medical still image appears to move in the direction corresponding to the movement in the horizontal direction of the imaging device 106 on the display screen”. The “medical still image appearing to move in the direction corresponding to the movement in the horizontal direction of the imaging device 106 on the display screen” refers to, for example, “the medical still image appearing to move in the up, down, left, and right directions (including diagonal directions) on the display screen in correspondence with the movement in the horizontal direction of the imaging device 106”.

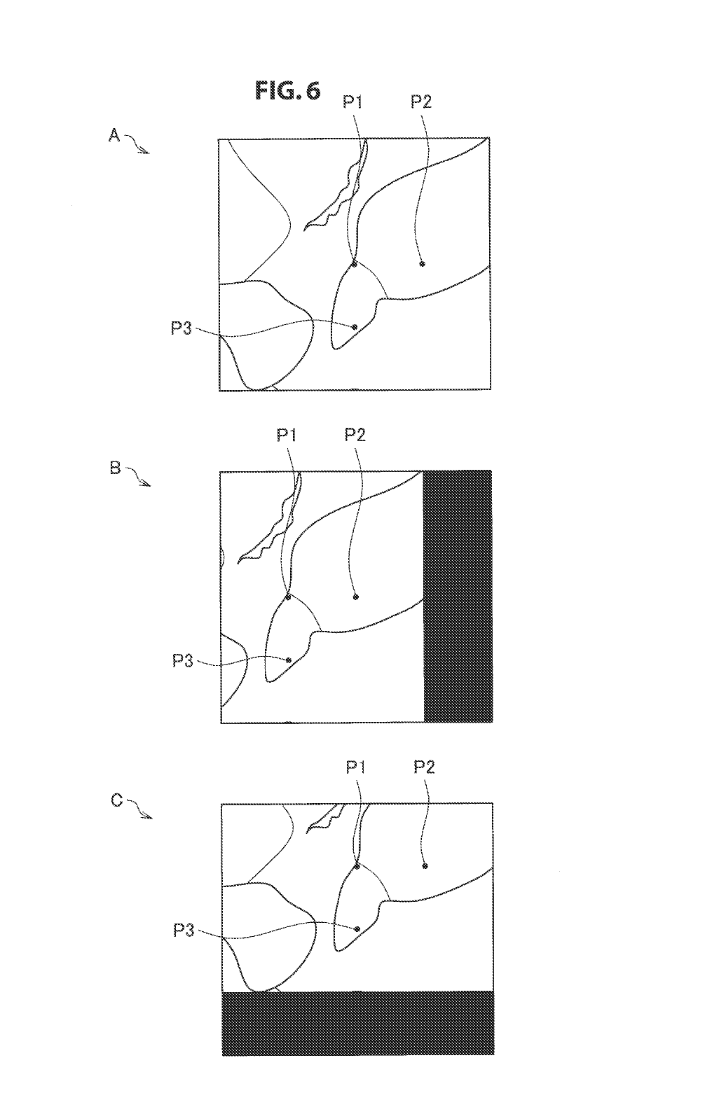

FIG. 6 is an explanatory diagram for explaining the first example of the processes related to the control method according to the present embodiment, and illustrates one example of the display of the medical still image changed in correspondence with the movement in the horizontal direction of the imaging device 106. A of FIG. 6 illustrates “one example of the medical still image being displayed on the display screen before the imaging device 106 moves in the horizontal direction”. B of FIG. 6 and C of FIG. 6 each illustrates “one example of the medical still image to be displayed on the display screen after the imaging device 106 has moved in the horizontal direction”.

For example, in the case in which the imaging device 106 moves from the position corresponding to a point P1 illustrated in A of FIG. 6 to the position corresponding to a point P2 illustrated in A of FIG. 6, the medical observation apparatus 100 alters the display of the medical still image such that the medical still image appears to move to the right on the display screen (B of FIG. 6).

Also, for example, in the case in which the imaging device 106 moves from the position corresponding to the point P1 illustrated in A of FIG. 6 to the position corresponding to a point P3 illustrated in A of FIG. 6, the medical observation apparatus 100 alters the display of the medical still image such that the medical still image appears to move downward on the display screen (C of FIG. 6).

The medical observation apparatus 100 specifies the movement of the imaging device 106 on the basis of a change in the rotational angle in each of the six rotation axes (one example of predetermined rotation axes) in the medical observation apparatus 100. If the change in the rotational angle of each of the six rotation axes is understood, the change in the shape of the arm 104 can be specified, and the movement direction and movement amount of the imaging device 106 supported by the arm 104 can be estimated from the change in the shape of the arm. Note that the method of specifying the movement of the imaging device 106 is not limited to the example illustrated above, and the medical observation apparatus 100 may also specify the movement of the imaging device 106 according to any method that enables the movement direction and the movement amount of the imaging device 106 to be specified (or estimated).

The movement direction of the imaging device 106 may be one or both of the horizontal direction of the imaging device 106 and the vertical direction of the imaging device 106. Movement in the horizontal direction of the imaging device 106 refers to, for example, “the imaging device 106 moving in a state such that the height of the imaging device 106 with respect to a reference plane such as the floor on which the medical observation apparatus 100 is installed (hereinafter simply designated the ‘height of the imaging device 106’) does not change”. Also, movement in the vertical direction of the imaging device 106 refers to, for example, “the imaging device 106 moving such that only the height of the imaging device 106 changes”. By combining movement in the horizontal direction of the imaging device 106 with movement in the vertical direction of the imaging device 106, the imaging device 106 is able to move anywhere inside the working range of the arm 104.

For example, in the case in which movement in the horizontal direction of the imaging device 106 is specified, by altering the position of “the region to display on the display screen in the medical still image” in correspondence with the movement in the horizontal direction of the imaging device 106, the medical observation apparatus 100 realizes a display of the medical still image like the examples illustrated in B of FIG. 6 and C of FIG. 6. By altering the position of the “region to display on the display screen in the medical still image”, a medical still image including a region where no image exists (illustrated as a black-filled region in the examples in FIG. 6) is displayed on the display screen. Note that examples of the medical still image displayed on the display screen after the imaging device 106 moves in the horizontal direction obviously are not limited to the examples illustrated in B of FIG. 6 and C of FIG. 6.

For example, in the case in which movement in the vertical direction of the imaging device 106 is specified, the medical observation apparatus 100 blurs the medical still image in correspondence with the movement in the vertical direction of the imaging device 106. By blurring the medical still image, the user or the like can be notified visually that the imaging device 106 has moved in the vertical direction. The medical observation apparatus 100 blurs the medical still image in correspondence with the movement in the vertical direction of the imaging device 106 by “executing a filter process applying coefficients according to the movement amount in the vertical direction of the imaging device 106 on the medical still image”, for example. Also, the medical observation apparatus 100 may blur the medical still image in correspondence with the movement in the vertical direction of the imaging device 106 by “selecting a filter corresponding to the movement amount in the vertical direction of the imaging device 106, and executing a filter process using the selected filter on the medical still image”, for example. Examples of the filter process according to the present embodiment include processes that use any filter capable of obtaining an image-blurring effect, such as an averaging filter or a Gaussian filter, for example. Note that the process of blurring the medical still image is not limited to a filter process, and the medical observation apparatus 100 may also blur the medical still image by executing the operations of any algorithm capable of obtaining an image-blurring effect on the medical still image.

Note that the process in the case of specifying movement in the vertical direction of the imaging device 106 is not limited to the example illustrated above.

For example, the medical observation apparatus 100 additionally may cause a notification image indicating movement in the vertical direction of the imaging device 106 to be displayed on the display screen in correspondence with the movement in the vertical direction of the imaging device 106. By causing the notification image to be displayed, the user or the like can be notified visually that the imaging device 106 has moved in the vertical direction. The medical observation apparatus 100 may cause the notification image to be displayed without blurring the medical still image in correspondence with the movement in the vertical direction of the imaging device 106, or cause the notification image to be displayed while also blurring the medical still image in correspondence with the movement in the vertical direction of the imaging device 106.

The notification image according to the present embodiment may be, for example, an indicator (for example, an indicator indicating the movement direction and the movement amount) that indicates movement in the vertical direction of the imaging device 106. Additionally, the notification image according to the present embodiment may indicate the movement in the vertical direction of the imaging device 106 by one or both of a text string indicating that the imaging device 106 has moved in the vertical direction, and an icon indicating that the imaging device 106 has moved in the vertical direction.

The medical observation apparatus 100 causes the notification image to be displayed additionally on the display screen by alpha-blending the notification image onto the medical still image, for example. Note that the method of causing the notification image to be displayed on the display screen is not limited to the example illustrated above, and the medical observation apparatus 100 may cause the notification image to be displayed on the display screen by any display control, such as picture-on-picture (POP).

As above, in the case in which the state related to the movement of the imaging device 106 changes from the first state to the second state, the medical observation apparatus 100 causes the medical still image to be displayed on the display screen. Additionally, like in the example illustrated in FIG. 6, for example, the medical observation apparatus 100 alters the display of the medical still image displayed on the display screen in correspondence with the movement of the imaging device 106.

The medical still image is a still image, and the display of the medical still image is altered in correspondence with the movement of the imaging device 106 by display control. Therefore, a person looking at the display on the display screen, such as a user, is able to visually recognize the movement of the imaging device 106 in a state with a reduced possibility of perceiving shake (image shake) in the medical still image.

Therefore, since the medical observation apparatus 100 is able to avoid situations like the above that may occur in association with the movement of the imaging device 106, convenience potentially may be improved for the user who uses the medical observation apparatus 100.

Note that the processes related to the control method according to the first example are not limited to the example illustrated above.

For example, in the case in which the state related to the movement of the imaging device 106 changes from the second state in which the imaging device 106 is movable to the first state in which the imaging device 106 is immovable, the medical observation apparatus 100 causes a medical captured image captured after the change from the second state to the first state to be displayed on the display screen. In other words, when triggered by the transition from the second state to the first state, the medical observation apparatus 100 switches the image displayed on the display screen from the medical still image to the real-time medical captured image being captured by the imaging device 106.

As described above, the medical observation apparatus 100 specifies the state related to movement on the basis of a result of detecting a predetermined switching operation, such as an operation on the operating mode change switch 128. In other words, the medical observation apparatus 100 treats an intentional operation by the user of the medical observation apparatus 100 such as the surgeon as a trigger, and switches the image displayed on the display screen from the medical still image to the real-time medical captured image.

At this point, when switching from the medical still image to the real-time medical captured image, the state related to movement is the first state in which the imaging device 106 is immovable. For this reason, when switching from the medical still image to the real-time medical captured image, situations like the above that may occur in association with the movement of the imaging device 106 do not occur. In other words, there is no reduction in convenience caused by the occurrence of situations like the above.

### (2) Second Example of Processes Related to Control Method

In the case in which the state related to the movement of the imaging device 106 changes from the first state in which the imaging device 106 is immovable to the second state in which the imaging device 106 is movable, the medical observation apparatus 100 causes a still image of the medical captured image captured by lowering the enlargement ratio when changing from the first state to the second state to be displayed on the display screen as the medical still image.

Also, when causing a still image of the medical captured image captured by lowering the enlargement ratio to be displayed as the medical still image, the medical observation apparatus 100 causes “an object indicating the capture range that had been captured before lowering the enlargement ratio” to be displayed.

Examples of the still image of the medical captured image captured by lowering the enlargement ratio when changing from the first state to the second state include the examples illustrated below.
A still image captured by the imaging device 106 by lowering the enlargement ratio at the point in time when the predetermined switching operation is detected
A frame image included in a moving image captured by the imaging device 106 by lowering the enlargement ratio at the point in time when the predetermined switching operation is detected
A still image captured by the imaging device 106 by lowering the enlargement ratio after the predetermined switching operation is detected but before movement of the imaging device 106 is detected
A frame image included in a moving image captured by the imaging device 106 by lowering the enlargement ratio after the predetermined switching operation is detected but before movement of the imaging device 106 is detected

The still image of the medical captured image captured by the imaging device 106 by lowering the enlargement ratio may be, for example, “a still image of the medical captured image captured by adjusting the optical system 120a included in the imaging member 120”. Also, in the case in which the imaging member 120 included in the imaging device 106 includes an imaging device that functions as the sub-camera described above, the still image of the medical captured image captured by the imaging device 106 by lowering the enlargement ratio may also be a still image of the medical captured image captured by the sub-camera.

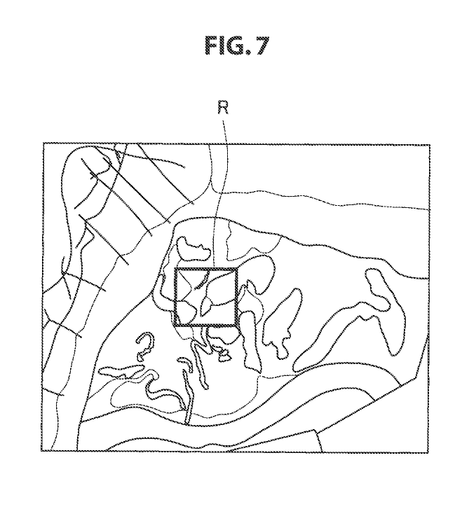

FIG. 7 is an explanatory diagram for explaining the second example of the processes related to the control method according to the present embodiment, and illustrates one example of a medical still image according to the second example of the processes related to the control method.

As illustrated in FIG. 7 for example, the medical observation apparatus 100 causes an object R indicating the capture range that had been captured before lowering the enlargement ratio to be displayed on top of the medical still image.

In the case in which the imaging device 106 moves in the horizontal direction, the medical observation apparatus 100 alters the display position of the object R in correspondence with the movement in the horizontal direction of the imaging device 106. The medical observation apparatus 100 alters the display position of the object R such that the object R appears to move in the up, down, left, and right directions in correspondence with the movement in the horizontal direction of the imaging device 106 on the display screen.

In the case in which the imaging device 106 moves in the vertical direction, the medical observation apparatus 100 blurs the medical still image in correspondence with the movement in the vertical direction of the imaging device, similarly to the processes related to the control method according to the first example above. Also, in the case in which the imaging device 106 moves in the vertical direction, the medical observation apparatus 100 additionally may cause a notification image to be displayed on the display screen, similarly to the processes related to the control method according to the first example above.

As illustrated in FIG. 7 for example, in the case in which the state related to the movement of the imaging device 106 changes from the first state to the second state, the medical observation apparatus 100 causes a still image of the medical captured image captured by lowering the enlargement ratio to be displayed on the display screen together with an object that indicates that capture range that had been captured before lowering the enlargement ratio. Also, the medical observation apparatus 100 alters the display position of the above object indicating the capture range in correspondence with the movement in the horizontal direction of the imaging device 106.

The medical still image is a still image, and the display position of the object indicating the capture range that had been captured before lowering the enlargement ratio is altered in correspondence with the movement of the imaging device 106. Therefore, a person looking at the display on the display screen, such as a user, is able to visually recognize the movement of the imaging device 106 in a state with a reduced possibility of perceiving shake (image shake) in the medical still image.

Therefore, since the medical observation apparatus 100 is able to avoid situations like the above that may occur in association with the movement of the imaging device 106, convenience potentially may be improved for the user who uses the medical observation apparatus 100.

Note that the processes related to the control method according to the second example are not limited to the example illustrated above.

For example, in the case in which the state related to the movement of the imaging device 106 changes from the second state in which the imaging device 106 is movable to the first state in which the imaging device 106 is immovable, similarly to the processes related to the control method according to the first example, the medical observation apparatus 100 may cause a medical captured image captured after the change from the second state to the first state to be displayed on the display screen.

### (3) Third Example of Processes Related to Control Method

In the case in which the state related to the movement of the imaging device 106 changes from the first state in which the imaging device 106 is immovable to the second state in which the imaging device 106 is movable, the medical still image that the medical observation apparatus 100 causes to be displayed is not limited to the medical still image in the processes related to the first control method and the medical still image in the processes related to the second control method.

For example, in the case in which the processes related to the first control method are executed, a medical still image including a region where no image exists as illustrated in FIG. 6 is displayed on the display screen. In contrast, the medical observation apparatus 100 that executes the processes related to the third control method interpolates the region where no image exists as illustrated in FIG. 6 on the basis of the medical captured image captured by lowering the enlargement ratio as illustrated in FIG. 7. In other words, in the case in which the processes related to the third control method are executed, a medical still image that does not include a region where no image exists as illustrated in FIG. 6 is displayed on the display screen.

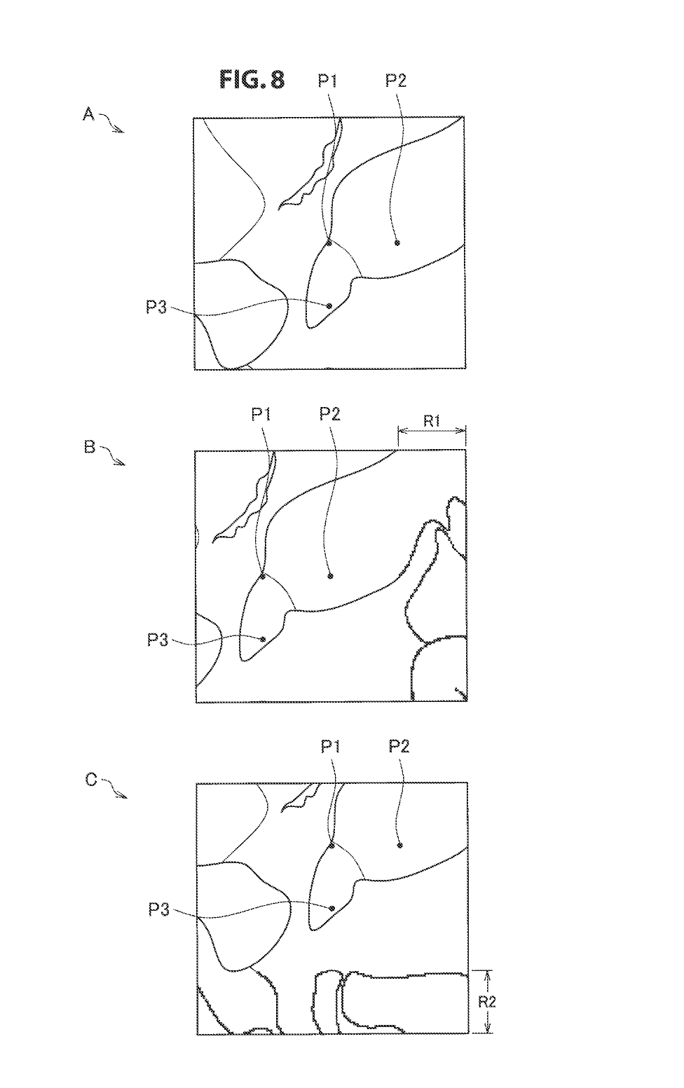

FIG. 8 is an explanatory diagram for explaining the third example of the processes related to the control method according to the present embodiment, and illustrates one example of the display of the medical still image changed in correspondence with the movement in the horizontal direction of the imaging device 106. A of FIG. 8 illustrates “one example of the medical still image being displayed on the display screen before the imaging device 106 moves in the horizontal direction”. B of FIG. 8 and C of FIG. 8 each illustrates “one example of the medical still image to be displayed on the display screen after the imaging device 106 has moved in the horizontal direction”.

For example, in the case in which the imaging device 106 moves from the position corresponding to a point P1 illustrated in A of FIG. 8 to the position corresponding to a point P2 illustrated in A of FIG. 8, the medical observation apparatus 100 alters the display of the medical still image such that the medical still image appears to move to the right on the display screen (B of FIG. 8).

In the case in which the imaging device 106 moves in the horizontal direction, similarly to the processes related to the first control method, the medical observation apparatus 100 alters the display of the medical still image such that the medical still image appears to move to the right by altering the position of the “region to be displayed on the display screen in the medical still image”. At this point, in the case of altering the position of the “region to be displayed on the display screen in the medical still image”, a region where no image exists is included as illustrated in B of FIG. 6.

Accordingly, the medical observation apparatus 100 interpolates the regional part where no image exists on the basis of the medical captured image captured by lowering the enlargement ratio. The medical captured image captured by lowering the enlargement ratio that is used for interpolation is acquired similarly to the processes related to the second control method, for example. From the medical captured image captured by lowering the enlargement ratio, the medical observation apparatus 100 specifies a region corresponding to the region where no image exists in the medical still image. For example, the medical observation apparatus 100 specifies a region corresponding to the region where no image exists in the above medical still image by any image matching process such as matching by detecting feature points. Subsequently, the medical observation apparatus 100 enlarges the specified regional part by electronic zoom, and interpolates the above regional part where no image exists with the enlarged image. Note that an example of the process of interpolating the regional part where no image exists obviously is not limited to the example illustrated above.

Also, for example, in the case in which the imaging device 106 moves from the position corresponding to the point P1 illustrated in A of FIG. 8 to the position corresponding to a point P3 illustrated in A of FIG. 8, the medical observation apparatus 100 alters the display of the medical still image such that the medical still image appears to move downward on the display screen (C of FIG. 8). The medical observation apparatus 100 executes a process similar to the above “example of altering the display of the medical still image such that the medical still image appears to move to the right”.

By executing the processes related to the third control method, for example, a regional part where no image exists, as illustrated by the region R1 illustrated in B of FIG. 8 and the region R2 illustrated in C of FIG. 8, is interpolated, and as a result, a medical still image that does not include a region where no image exists is displayed on the display screen.

In the case in which the imaging device 106 moves in the vertical direction, the medical observation apparatus 100 blurs the medical still image in correspondence with the movement in the vertical direction of the imaging device, similarly to the processes related to the control method according to the first example above. Also, in the case in which the imaging device 106 moves in the vertical direction, the medical observation apparatus 100 additionally may cause a notification image to be displayed on the display screen, similarly to the processes related to the control method according to the first example above.

The processes related to the third control method basically are similar to the processes related to the first control method above, except that the regional part where no image exists is interpolated. Therefore, in the medical observation apparatus 100 that executes the processes related to the third control method, similarly to the medical observation apparatus 100 that executes the processes related to the first control method above, convenience potentially may be improved for the user who uses the medical observation apparatus 100.

Note that the processes related to the control method according to the third example are not limited to the example illustrated above.

For example, in the case in which the state related to the movement of the imaging device 106 changes from the second state in which the imaging device 106 is movable to the first state in which the imaging device 106 is immovable, similarly to the processes related to the control method according to the first example, the medical observation apparatus 100 may cause a medical captured image captured after the change from the second state to the first state to be displayed on the display screen.

### [2-3] Specific Example of Processes Related to Control Method According to Present Embodiment

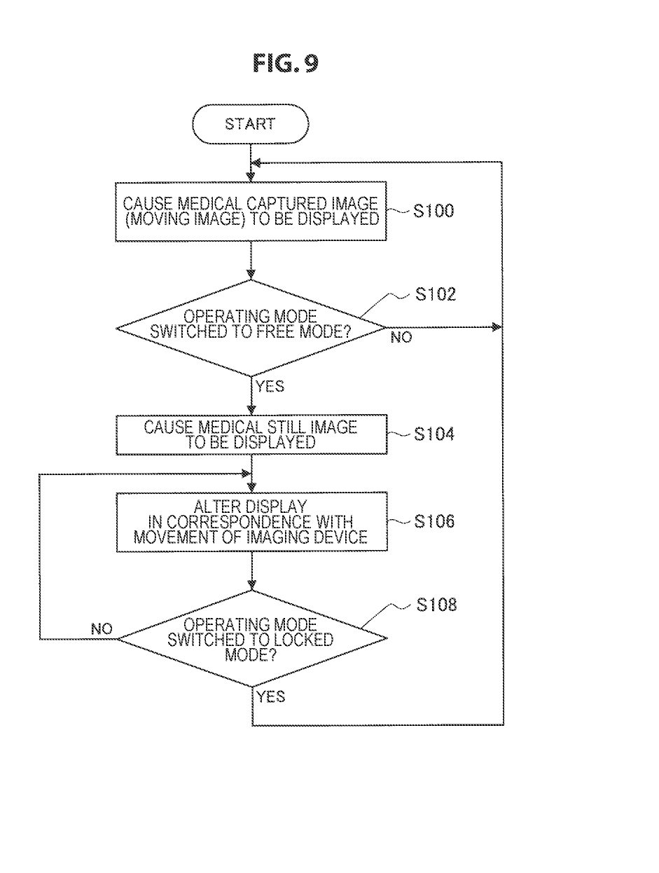

FIG. 9 is a flowchart illustrating one example of processes related to the control method according to the present embodiment.

The medical observation apparatus 100 causes a medical captured image (moving image) to be displayed on the display screen (S100). The medical captured image displayed on the display screen in step S100 is a real-time medical captured image being captured by the imaging device 106.

The medical observation apparatus 100 determines whether or not the operating mode of the arm 104 has switched to the free mode (S102). For example, the medical observation apparatus 100 determines whether or not the operating mode has switched to the free mode on the basis of an operation on the operating mode change switch 128. The process in step S102 corresponds to a process of determining whether or not the state related to the movement of the imaging device 106 has changed from the first state in which the imaging device 106 is immovable to the second state in which the imaging device 106 is movable.

In the case in which a switch to the free mode is not determined in step S102, the medical observation apparatus 100 repeats the processes from step S100.

Also, in the case in which a switch to the free mode is determined in step S102, the medical observation apparatus 100 causes a medical still image to be displayed on the display screen (S104). Additionally, the medical observation apparatus 100 alters the display of the medical still image in correspondence with the movement of the imaging device 106 (S106). The medical observation apparatus 100 alters the display of the medical still image according to any one of the processes related to the first example illustrated in (1) described above to the processes related to the third example illustrated in (3) described above.

The medical observation apparatus 100 determines whether or not the operating mode of the arm 104 has switched to the locked mode (S108). For example, the medical observation apparatus 100 determines whether or not the operating mode has switched to the locked mode on the basis of an operation on the operating mode change switch 128. The process in step S108 corresponds to a process of determining whether or not the state related to the movement of the imaging device 106 has changed from the second state in which the imaging device 106 is movable to the first state in which the imaging device 106 is immovable.

In the case in which a switch to the locked mode is not determined in step S108, the medical observation apparatus 100 repeats the processes from step S106. Also, in the case in which a switch to the locked mode is determined in step S108, the medical observation apparatus 100 repeats the processes from step S100.

The medical observation apparatus 100 executes the processes illustrated in FIG. 9 for example as the processes related to the control method according to the present embodiment. Note that an example of the processes related to the control method according to the present embodiment obviously is not limited to the example illustrated in FIG. 9.

### [3] Example of Advantageous Effects Exhibited by Use of Control Method According to Present Embodiment

By using the control method according to the present embodiment, the advantageous effects illustrated below are exhibited, for example. Note that the advantageous effects exhibited by using the control method according to the present embodiment obviously are not limited to the examples illustrated below.
When an operation of releasing the brake on the arm 104, such as an operation of switching to the free mode on the operating mode change switch 128, is detected, the medical observation apparatus 100 switches the medical captured image displayed on the display screen of the display apparatus 200 from a moving image to a still image. Also, the medical observation apparatus 100 alters the display of the still image being displayed on the basis of the motion of the arm 104 that supports the imaging device 106. At this point, since the arm 104 supports the imaging device 106, altering the display of the still image on the basis of the motion of the arm 104 corresponds to altering the display of the still image in correspondence with the movement of the imaging device 106. Therefore, a person looking at the display on the display screen, such as a user of the medical observation apparatus 100, is able to visually recognize the movement of the imaging device 106 in a state with a reduced possibility of perceiving shake (image shake) in the medical captured image being displayed on the display screen.
By “visually recognizing the movement of the imaging device 106 in a state with a reduced possibility of perceiving shake (image shake) in the medical captured image being displayed on the display screen”, the user of the medical observation apparatus 100 is able to “move the imaging device 106 such that a desired capture range is captured while looking at the medical captured image displayed on the display screen”, even in the case in which the imaging device 106 is imaging at a high magnification.
By “visually recognizing the movement of the imaging device 106 in a state with a reduced possibility of perceiving shake (image shake) in the medical captured image being displayed on the display screen”, dizziness and disorientation caused by the shaking of the medical captured image that the user of the medical observation apparatus 100 may possibly perceive can be reduced.

### (Program According to Present Embodiment)

By having a program (for example, a program capable of executing the processes related to the control method according to the present embodiment) for causing a computer system to function as the medical observation apparatus according to the present embodiment (or the control apparatus according to the present embodiment) be executed by a processor or the like in the computer system, convenience for the user who uses the medical observation system can be improved. Herein, the computer system according to the present embodiment may be a single computer or multiple computers. A series of processes related to the control method according to the present embodiment is executed by the computer system according to the present embodiment.

Additionally, by having the program for causing a computer system to function as the medical observation apparatus according to the present embodiment (or the control apparatus according to the present embodiment) be executed by a processor or the like in the computer system, the advantageous effects exhibited by the display realized by the processes related to the control method according to the present embodiment described above can be exhibited.

It should be understood by those skilled in the art that various modifications, combinations, sub-combinations and alterations may occur depending on design requirements and other factors insofar as they are within the scope of the appended claims or the equivalents thereof.

For example, although the above illustrates the provision of a program (computer program) for causing a computer system to function as the medical observation apparatus according to the present embodiment, in the present embodiment, the above program may also be provided in conjunction with a recording medium on which the above program is stored.

The configuration described above illustrates one example of the present embodiment, and rightfully belongs to the technical scope of the present disclosure.

Further, the effects described in this specification are merely illustrative or exemplified effects, and are not limitative. That is, with or in the place of the above effects, the technology according to the present disclosure may achieve other effects that are clear to those skilled in the art from the description of this specification.

Additionally, the present technology may also be configured as below.

(1) A medical control apparatus including:

a display control section configured to control a display of a medical captured image captured by an imaging device on the basis of a state related to a movement of the imaging device, in which

in a case in which the state related to the movement of the imaging device is a first state in which the imaging device is immovable, the display control section causes the medical captured image to be displayed on a display screen, and

in a case in which the state related to the movement of the imaging device is a second state in which the imaging device is movable, the display control section causes a medical still image to be displayed on the display screen, and changes a display of the display screen according to the movement of the imaging device.

(2) The medical control apparatus according to (1), in which

the display control section alters the display of the medical still image such that the medical still image appears to move in a direction corresponding to a movement in a horizontal direction of the imaging device on the display screen.

(3) The medical control apparatus according to (1) or (2), in which

the display control section alters a position of a region to be displayed on the display screen in the medical still image in correspondence with a movement in a horizontal direction of the imaging device.

(4) The medical control apparatus according to any one of (1) to (3), in which

the display control section blurs the medical still image in correspondence with a movement in a vertical direction of the imaging device.

(5) The medical control apparatus according to any one of (1) to (4), in which

the display control section additionally causes a notification image indicating a movement in a vertical direction of the imaging device to be displayed on the display screen in correspondence with the movement in the vertical direction.

(6) The medical control apparatus according to any one of (1) to (5), in which

the medical still image is a still image of a medical captured image captured when changing from the first state to the second state.

(7) The medical control apparatus according to (1), in which

the medical still image is a still image of a medical captured image captured by lowering an enlargement ratio when changing from the first state to the second state, and

the display control section causes an object indicating a capture range that had been captured before lowering the enlargement ratio to be displayed on the medical still image.

(8) The medical control apparatus according to (7), in which

the display control section alters a display position of the object in correspondence with a movement in a horizontal direction of the imaging device.

(9) The medical control apparatus according to any one of (1) to (8), in which

in a case of changing from the second state to the first state, the display control section causes a medical captured image captured after changing from the second state to the first state to be displayed on the display screen.

(10) The medical control apparatus according to any one of (1) to (9), in which

the display control section specifies the state related to the movement on the basis of a result of detecting a predetermined switching operation that switches between the first state and the second state.

(11) The medical control apparatus according to any one of (1) to (10), in which

the imaging device is supported by an arm including multiple links joined to each other by one or multiple joint sections, the arm being configured to be rotatable about one or multiple predetermined rotation axes, and

the display control section specifies a movement of the imaging device on the basis of a change of a rotational angle in the one or multiple predetermined rotation axes.

(12) The medical control apparatus according to any one of (1) to (11), including:

an arm including multiple links joined to each other by one or multiple joint sections; and

the imaging device supported by the arm.

(13) A medical observation apparatus including:

an arm including multiple links joined to each other by one or multiple joint sections; and

an imaging device supported by the arm; and

a display control section configured to control a display of a medical captured image captured by the imaging device on the basis of a state related to a movement of the imaging device, in which

in a case in which the state related to the movement of the imaging device is a first state in which the imaging device is immovable, the display control section causes the medical captured image to be displayed on a display screen, and

in a case in which the state related to the movement of the imaging device is a second state in which the imaging device is movable, the display control section causes a medical still image to be displayed on the display screen, and changes a display of the display screen according to the movement of the imaging device.

(14) A control method executed by a medical control apparatus, the control method including:

controlling a display of a medical captured image captured by an imaging device on the basis of a state related to a movement of the imaging device, in which

in a case in which the state related to the movement of the imaging device is a first state in which the imaging device is immovable, the controlling causes the medical captured image to be displayed on a display screen, and

in a case in which the state related to the movement of the imaging device is a second state in which the imaging device is movable, the controlling causes a medical still image to be displayed on the display screen, and changes a display of the display screen according to the movement of the imaging device.

What is claimed is:

1. A medical control apparatus comprising:
a display control section configured to control a display of a medical captured image captured by an imaging device on a basis of a state related to a movement of the imaging device, wherein
in a case in which the state related to the movement of the imaging device is a first state in which the imaging device is immovable, the display control section causes the medical captured image to be displayed on a display screen, and
in a case in which the state related to the movement of the imaging device is a second state in which the imaging device is movable, the display control section causes a medical still image to be displayed on the display screen, and changes a display of the display screen according to the movement of the imaging device.

2. The medical control apparatus according to claim 1, wherein
the display control section alters the display of the medical still image such that the medical still image appears to move in a direction corresponding to a movement in a horizontal direction of the imaging device on the display screen.

3. The medical control apparatus according to claim 1, wherein
the display control section alters a position of a region to be displayed on the display screen in the medical still image in correspondence with a movement in a horizontal direction of the imaging device.

4. The medical control apparatus according to claim 1, wherein
the display control section blurs the medical still image in correspondence with a movement in a vertical direction of the imaging device.

5. The medical control apparatus according to claim 1, wherein
the display control section additionally causes a notification image indicating a movement in a vertical direction of the imaging device to be displayed on the display screen in correspondence with the movement in the vertical direction.

6. The medical control apparatus according to claim 1, wherein
the medical still image is a still image of a medical captured image captured when changing from the first state to the second state.

7. The medical control apparatus according to claim 1, wherein
the medical still image is a still image of a medical captured image captured by lowering an enlargement ratio when changing from the first state to the second state, and
the display control section causes an object indicating a capture range that had been captured before lowering the enlargement ratio to be displayed on the medical still image.

8. The medical control apparatus according to claim 7, wherein
the display control section alters a display position of the object in correspondence with a movement in a horizontal direction of the imaging device.

9. The medical control apparatus according to claim 1, wherein
in a case of changing from the second state to the first state, the display control section causes a medical captured image captured after changing from the second state to the first state to be displayed on the display screen.

10. The medical control apparatus according to claim 1, wherein
the display control section specifies the state related to the movement on a basis of a result of detecting a predetermined switching operation that switches between the first state and the second state.

11. The medical control apparatus according to claim 1, wherein
the imaging device is supported by an arm including multiple links joined to each other by one or multiple joint sections, the arm being configured to be rotatable about one or multiple predetermined rotation axes, and
the display control section specifies a movement of the imaging device on a basis of a change of a rotational angle in the one or multiple predetermined rotation axes.

12. The medical control apparatus according to claim 1, comprising:
an arm including multiple links joined to each other by one or multiple joint sections; and
the imaging device supported by the arm.

13. A medical observation apparatus comprising:
an arm including multiple links joined to each other by one or multiple joint sections; and
an imaging device supported by the arm; and
a display control section configured to control a display of a medical captured image captured by the imaging device on a basis of a state related to a movement of the imaging device, wherein
in a case in which the state related to the movement of the imaging device is a first state in which the imaging device is immovable, the display control section causes the medical captured image to be displayed on a display screen, and
in a case in which the state related to the movement of the imaging device is a second state in which the imaging device is movable, the display control section causes a medical still image to be displayed on the display screen, and changes a display of the display screen according to the movement of the imaging device.

14. A control method executed by a medical control apparatus, the control method comprising:
controlling a display of a medical captured image captured by an imaging device on a basis of a state related to a movement of the imaging device, wherein
in a case in which the state related to the movement of the imaging device is a first state in which the imaging device is immovable, the controlling causes the medical captured image to be displayed on a display screen, and
in a case in which the state related to the movement of the imaging device is a second state in which the imaging device is movable, the controlling causes a medical still image to be displayed on the display screen, and changes a display of the display screen according to the movement of the imaging device.
